(19) United States
(12) Patent Application Publication
(10) Pub. No.: US 2009/0187134 A1
(43) Pub. Date: Jul. 23, 2009

(54) Iontophoresis Device Controlling Amounts of a Sleep-Inducing Agent and a Stimulant to be Administered and Time at Which the Drugs are Administered

(76) Inventors: Hidero Akiyama, Shibuya-ku (JP); Mizuo Nakayama, Shibuya-ku (JP); Takehiko Matsumura, Shibuya-ku (JP); Akihiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 11/992,673

(22) PCT Filed: Oct. 2, 2006
(86) PCT No.: PCT/JP2006/319685

§ 371 Date: Mar. 20, 2009

(30) Foreign Application Priority Data
Sep. 30, 2005 (JP) 2005-287248

(51) Int. Cl. A61N 1/30

(52) U.S. Cl. 604/20

(57) ABSTRACT

An iontophoresis device includes an electric power source device, a drug administration device and a current control device. The drug administration device may include at least two or more electrode assemblies each holding an ionic drug. The drug administration device may be coupled to the electric power source device. The current control device may control current flowing to respective ones of the electrode assemblies. An amount of the ionic drug is releasable from each of the electrode assemblies at a defined time when transdermally administered to an organism in accordance with the current flowing from the current control device, wherein at least one of the two or more electrode assemblies holds a sleep-inducing agent as the ionic drug, and at least another one of the two or more electrode assemblies holds a stimulant as the ionic drug.

### BACKGROUND

1. Technical Field

The present disclosure relates to a technique for transdermally administering various ionic drugs (transdermal drug delivery) by means of iontophoresis. In particular, the present disclosure relates to an iontophoresis device for administering each of a sleep-inducing agent and a stimulant to an organism while individually controlling the amount of each to be administered and the time at which each is administered.

2. Description of the Related Art

A method of introducing (permeating) an ionic drug placed on the surface of the skin or mucosa (hereinafter, merely referred to as “skin”) of a predetermined site of an organism into the body through the skin by giving the skin an electromotive force sufficient to drive such an ionic drug is called iontophoresis (iontophorese, ion introduction method, ion permeation therapy) (See e.g., JP 63-35266 A).

For example, positively charged ions are driven (transported) into the skin on the side of an anode (positive electrode) in an electric system of an iontophoresis device. On the other hand, negatively charged ions are driven (transported) into the skin on the side of a cathode (negative electrode) in the electric system of the iontophoresis device.

Conventionally, a large number of such iontophoresis devices as described above have been proposed (See e.g., 63-35266 A and WO 03/037425 A1).

Such conventional iontophoresis devices as described above qualifies, in principle, for the transdermal administration of a single drug. It should be noted that the administration of only one kind of sleep-inducing agent as a drug by means of an iontophoresis device may involve the emergence of a problem of poor awakening. The problem can be alleviated by administering a stimulant as another drug at the time of awakening.

Therefore, it is desirable to enable the control of: the amount of each of two or more kinds of drugs to be administered, for example, a sleep-inducing agent and a stimulant; and the time at which each of the two or more kinds of drugs is administered in an iontophoresis device.

### BRIEF SUMMARY

Embodiments of the present invention provide an iontophoresis device enabling the control of: the amount of each of a sleep-inducing agent and a stimulant to be administered; and the time at which each is administered.

According to one embodiment, an iontophoresis includes an electric power source device, drug administration device including at least two or more electrode assemblies each holding an ionic drug, the drug administration device coupled to the electric power source device, and current control device to control current flowing to respective ones of the electrode assemblies, an amount of the ionic drug is releasable from each of the electrode assemblies at a defined time when transdermally administered to an organism in accordance with the current flowing from the current control device, wherein at least one of the two or more electrode assemblies holds a sleep-inducing agent as the ionic drug, and at least another one of the two or more electrode assemblies holds a stimulant as the ionic drug.

According to one embodiment, a method of operating an iontophoresis device includes providing an electric power source device, coupling a drug administration device to the electric power source device, the drug administration device including at least two or more electrode assemblies respectively holding a sleep-inducing drug and a stimulant drug, placing the drug administration device on a skin surface of an organism, energizing the drug administration device with the electric power source device, providing a current control device to control current flowing through respective ones of the electrode assemblies, and releasing a defined amount of the sleep-inducing drug and the stimulant drug at a defined time.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 shows a bottom view of an iontophoresis device according to one illustrated embodiment of the present invention.

FIG. 2 shows a sectional view of drug administration means in the iontophoresis device according to one illustrated embodiment of the present invention.

FIG. 3 shows a circuit diagram of the iontophoresis device according to one illustrated embodiment of the present invention.

### DETAILED DESCRIPTION

As described above, according to one embodiment of the present invention, current control means for individually controlling each of the currents flowing into multiple electrode assemblies separately holding a sleep-inducing agent and a stimulant as ionic drugs is installed in the iontophoresis device. Each of the electrode assemblies may be adapted to release an amount of ionic drug at a defined time in accordance with a current flowing from the current control means. As a result, each of the sleep-inducing agent and the stimulant may be administered while controlling the amount of each to be administered and the time at which each is administered.

As described above, the iontophoresis device according to one embodiment of the present invention may include: an electric power source device; drug administration means coupled to the electric power source device and including at least two or more electrode assemblies each holding an ionic drug; and current control means for individually controlling each of the currents flowing into the electrode assemblies. Each of the electrode assemblies may be adapted to release an amount of ionic drug at a defined time in accordance with a current flowing from the current control means so that the drug is transdermally administered to an organism 14. At least one of the two or more electrode assemblies, which each hold the ionic drug, holds a sleep-inducing agent as the ionic drug; and another at least one of the two or more electrode assemblies each holding the ionic drug holds a stimulant as the ionic drug.

Hereinafter, embodiments of the present invention will be described on the basis of specific examples shown in the drawings.

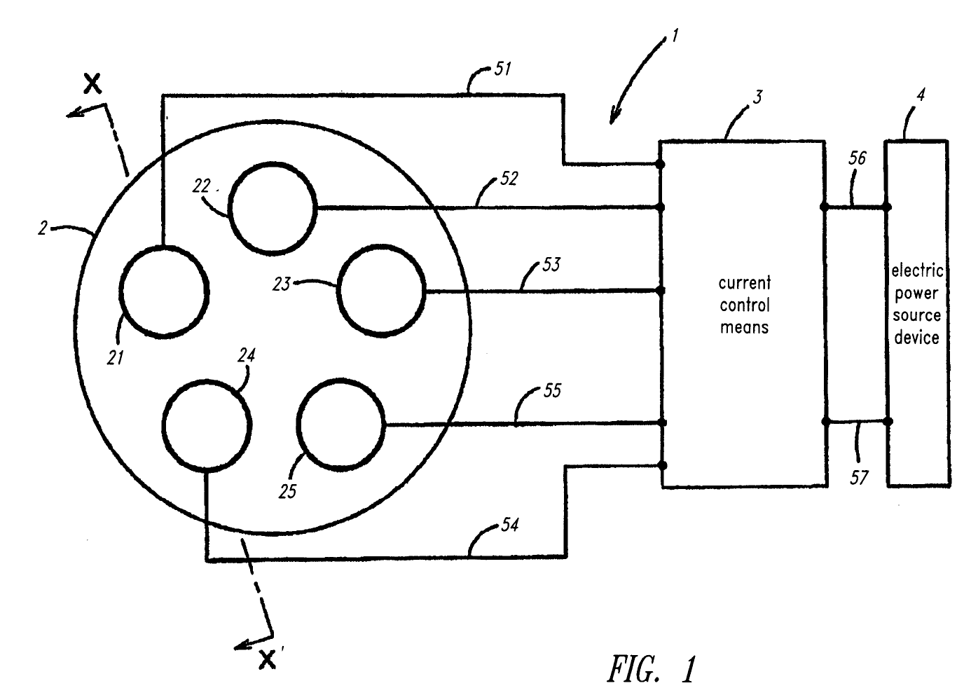

FIG. 1 shows a bottom view of an iontophoresis device 1, according to one illustrated embodiment. The iontophoresis device 1 includes a drug administration means 2 mounted on a skin 6 of the organism 14, current control means 3, and an electric power source device 4. The drug administration means 1 includes multiple electrode assemblies 21, 22, 23, 24, and 25. In addition, first electrode assemblies 21, 22, and 23 from the multiple electrode assemblies 21, 22, 23, 24, and 25 in the drug administration means 2 are coupled to the current control means 3 via electric wires 51, 52, and 53, and second electrode assemblies 24 and 25. The second electrode assemblies 24 and 25 serve as counter electrodes of the first electrode assemblies 21, 22, and 23 and are coupled to the current control means 3 via electric wires 54 and 55. Furthermore, the current control means 3 is coupled to the electric power source device 4 via electric wires 56 and 57. In one embodiment, one of the second electrode assemblies 24 and 25 may be arranged. According to that embodiment, the second electrode assembly 25 and the electric wire 55 are removed, and the second electrode assembly 24 is coupled to the current control means 3 via the electric wire 54.

According to the above specific example, the multiple electrode assemblies 21, 22, 23, 24, and 25 in the drug administration means 2 are gathered in one package to be constituted integrally. The multiple electrode assemblies 21, 22, 23, 24, and 25 may be constituted so as to be apart from one another. Alternatively, a portion of the multiple electrode assemblies 21, 22, 23, 24, and 25 may be gathered in one package.

In the above specific example, the drug administration means 2, the current control means 3, and the electric power source device 4 are arranged at positions apart from one another. For example, the following procedure may be adopted: the electric power source device 4 is constituted as a button battery and the current control means 3 is constituted as an integrated circuit for achieving size reduction, thereby constituting the drug administration means 2, the current control means 3, and the electric power source device 4 integrally.

In the drug administration means 2, according to one embodiment of the present invention, counter electrodes of an electrode assembly which hold a sleep-inducing agent and a stimulant as ionic drugs may be arranged so that each of the drugs can be administered alone. Alternatively, one counter electrode may be arranged when the sleep-inducing agent and the stimulant are ionized to have same polarity.

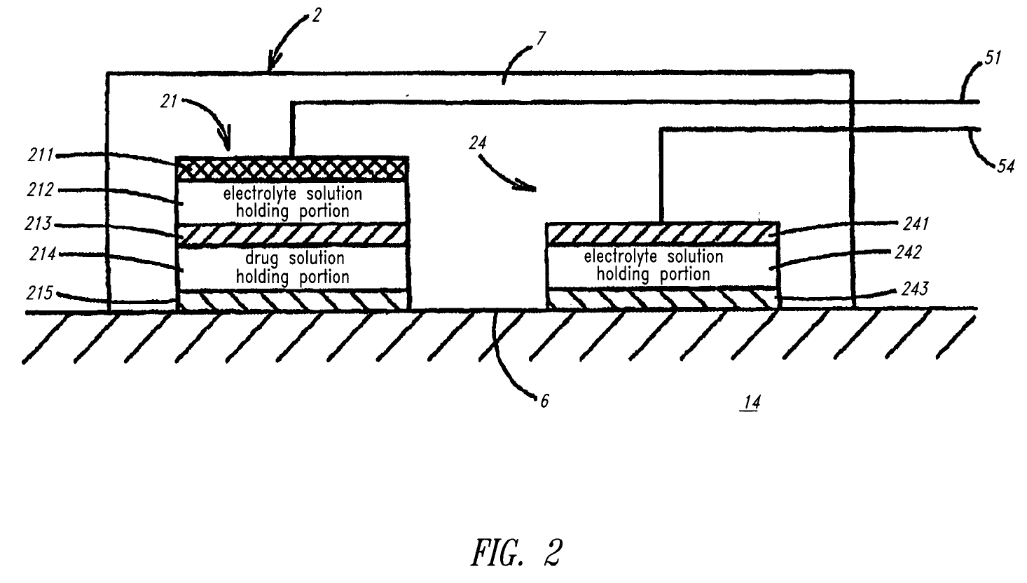

Description will be given of the case illustrated in FIG. 2, where the first electrode assembly 21 holds an ionic drug and the second electrode assembly 24 is a counter electrode that does not hold an ionic drug, by way of a specific constitution of an electrode assembly.

FIG. 2 is a cross-sectional view along X-X′ of the drug administration means 2 illustrated in FIG. 1, according to one illustrated embodiment. In the embodiment illustrated in FIG. 2, the drug administration means 2 is arranged on a skin 6. In addition, the electrode assemblies 21 and 24 are encased by one package 7.

The first electrode assembly 21 may include at least an electrode 211 coupled to a same polarity of the electric power source device 3 as that of a drug component of an ionic drug in the first electrode assembly 21 via the electric wire 51. An electrolyte solution holding portion 212 may hold an electrolyte solution by being impregnated with the electrolyte solution. The electrolyte solution holding portion 212 may be arranged adjacent to the electrode 211. An ion exchange membrane 213 may select an ion having polarity opposite that of a charged ion of the ionic drug. The ion exchange membrane 213 may be arranged adjacent to the electrolyte solution holding portion 212. A drug solution holding portion 214 may hold the ionic drug by being impregnated with the ionic drug. The drug solution holding portion 214 may be arranged adjacent to the ion exchange membrane 213. An ion exchange membrane 215 may select an ion having same polarity as that of the charged ion of the ionic drug. The ion exchange membrane 215 may be arranged adjacent to the drug solution holding portion 214.

The second electrode assembly 24 coupled to the electric power source device 3 via the electric wire 54 may include at least an electrode 241 opposite in polarity to the electrode 211 in the first electrode assembly 21. An electrolyte solution holding portion 242 to hold an electrolyte solution by being impregnated with the electrolyte solution The electrolyte solution holding portion 242 may be arranged adjacent to the electrode 241 An ion exchange membrane 243 to select an ion having polarity opposite that of the charged ion of the ionic drug in the first electrode assembly 21. The ion exchange membrane 243 may be arranged adjacent to the electrolyte solution holding portion 242.

According to one embodiment, the second electrode assembly 24 may be of same electrode structure as that of the above-described first electrode assembly 21 when the second electrode assembly 24 holds an ionic drug. According to another embodiment, even when the second electrode assembly 24 does not hold any ionic drug, the second electrode assembly 24 can be of the same electrode structure as that of the above-described first electrode assembly 21.

When the electrode assembly 21 holding the ionic drug is energized, the ionic drug in the drug solution holding portion 214 moves due to electrophoresis toward a side opposite the electrode 211 by virtue of an electric field, and is administered into the skin 6 via the ion exchange membrane 215. As such, the ion exchange membrane 213 arranged on the side of the electrode 211 may select an ion having polarity opposite that of a charged ion of the ionic drug, thereby preventing movement of the ionic drug toward the electrode 211. Meanwhile, the ion exchange membrane 215 arranged on the skin 6 may select an ion having the same polarity as that of the charged ion of the ionic drug, such that the ionic drug may be efficiently released, and may be administered into the skin at high transport efficiency. At the same time, an ion having a polarity opposite that of the ionic drug may be prevented from moving from the side of the organism 14 (e.g., skin 6) to the side of the drug solution holding portion 214. Movement of H⁺ or OH⁻ generated at the electrode 211 toward the skin 6 may also be suppressed such that a change in pH on the skin 6 may be suppressed. Furthermore, the electrode assembly 21 in embodiments of the present invention has such constitution as described above, thereby preventing damage to the skin 6 based on an electrochemical reaction and enabling the ionic drug to be safely administered.

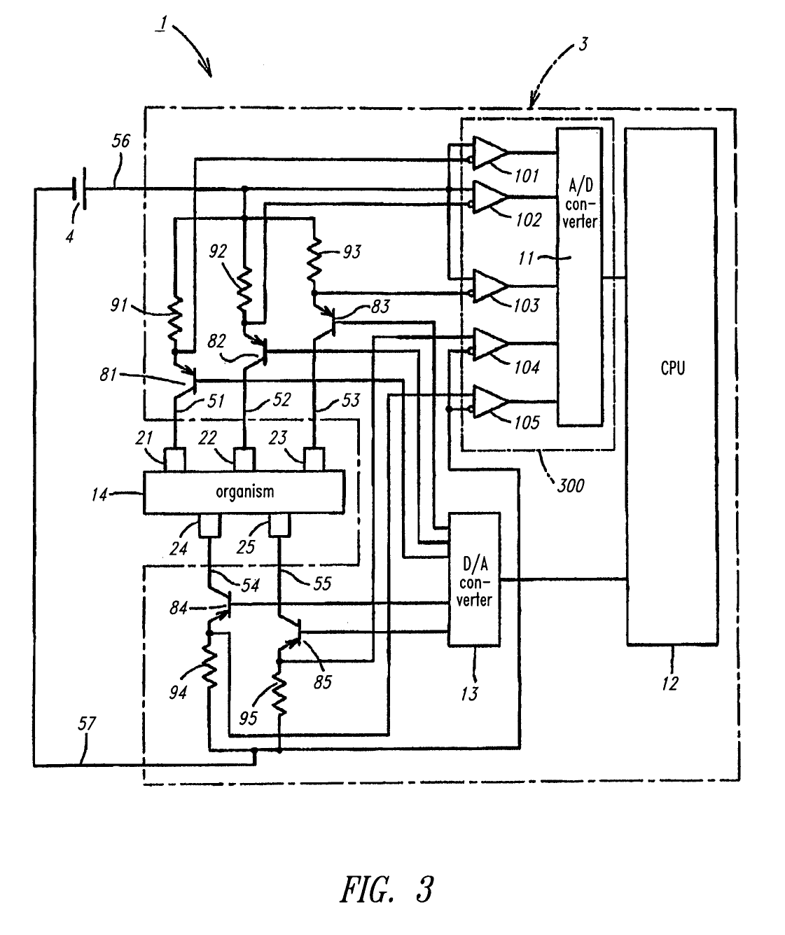

Next, a specific example of one embodiment of the current control means 3 of the iontophoresis device 1 will be described with reference to FIG. 3. The iontophoresis device 1 includes a circuit as illustrated in FIG. 3, and may enable an amount of ionic drug to be released at a defined time and enable control such that a current having a defined value flows into each electrode assembly 21, 22, 23, 24, and 25 holding an ionic drug irrespective of impedance and change with time of the skin 6.

As shown in FIG. 3, the current control means 3 in the iontophoresis device 1 may include load resistances 91, 92, 93, 94, and 95 arranged between the electrode assemblies 21, 22, 23, 24, and 25 and the electric power source device 4. Current detecting portions 101, 102, 103, 104, 105, and 11 may detect currents flowing into the load resistances 91, 92, 93, 94, and 95 Feedback control portions 12 and 13, and 81, 82, 83, 84, and 85 may cause controlled currents to flow into the electrode assemblies 21, 22, 23, 24, and 25 in accordance with outputs from the current detecting portions 101, 102, 103, 104, 105, and 11.

The current detecting portions 101, 102, 103, 104, 105, and 11 may take the form of current detection circuits 101, 102, 103, 104, and 105 detecting currents flowing into the load resistances 91, 92, 93, 94, and 95, and an A/D converter 11 for converting an output from each of the current detection circuits 101, 102, 103, 104, and 105 into a digital signal. The A/D converter 11 may output the digital signal to each of the feedback control portions 12 and 13, and 81, 82, 83, 84, and 85.

The above-described feedback control portions 12 and 13, and 81, 82, 83, 84, and 85 may take the form of a CPU 12 that outputs a feedback signal to each of the electrode assemblies 21, 22, 23, 24, and 25 in accordance with an output from each of the current detecting portions 101, 102, 103, 104, 105, and 11, a D/A converter 13 for converting the feedback signal into an analog signal, and transistors 81, 82, 83, 84, and 85 arranged between the electrode assemblies 21, 22, 23, 24, and 25 and the load resistances 91, 92, 93, 94, and 95 and causing controlled currents to flow into the electrode assemblies 21, 22, 23, 24, and 25 in accordance with outputs from the D/A converter 13. Emitters of the transistors 81, 82, 83, 84, and 85 may be coupled to the load resistances 91, 92, 93, 94, and 95, the bases of the transistors 81, 82, 83, 84, and 85 may be coupled to the D/A converter 13, and the collectors of the transistors 81, 82, 83, 84, and 85 may be coupled to the electrode assemblies 21, 22, 23, 24, and 25.

Differential amplifiers may be used for the current detection circuits 101, 102, 103, 104, and 105. Each of the differential amplifiers may detect a value for a voltage across each of the load resistances 91, 92, 93, 94, and 95. The amplifiers may detect current values from those voltage values and the resistance values of the above respective load resistances 91, 92, 93, 94, and 95.

In addition, the load resistances 91, 92, 93, 94, and 95 may be fixed resistances. The resistance values in the fixed resistances can be appropriately set on the basis of, for example, a defined value for a current to flow into each of the electrode assemblies 21, 22, 23, 24, and 25. The resistance values may each be approximately 10Ω or less in consideration of, for example, an influence on the operating state of the iontophoresis device 1.

Operation of the iontophoresis device 1 will be described below with reference to FIG. 3.

The current detection circuits 101, 102, 103, 104, and 105 detect currents flowing from the electric power source device 4 to the respective fixed resistances 91, 92, 93, 94, and 95. Signals in response to the detected currents are transmitted to the CPU 12 via the A/D converter 11. The CPU 12 performs predetermined data processing in response to a signal from the A/D converter 11, and sends a feedback signal to the D/A converter 13. The D/A converter 13 may cause a current to flow into at least one of the transistors 81, 82, 83, 84, and 85, in response to the feedback signal from the CPU 12. In accordance with the currents flowing from the transistors 81, 82, 83, 84, and 85, an amount of ionic drug may be released from each of the electrode assemblies 21, 22, and 23 at a defined time, such that the ionic drug may be transdermally administered to the organism 14.

The CPU 12 may have a built-in algorithm and may perform data processing based on the algorithm to output a feedback signal for causing each of the electrode assemblies 21, 22, 23, 24, and 25 to release a defined amount of ionic drug at a defined time. Accordingly, altering the algorithm of the CPU 12 may change the order in which currents flow into the respective electrode assemblies 21, 22, 23, 24, and 25, a time at which each of the currents flows, a combination of the respective electrode assemblies 21, 22, 23, 24, and 25, and the like.

Furthermore, the CPU 12 may perform control such that a current having a defined value flows into each of the electrode assemblies 21, 22, 23, 24, and 25 irrespective of the impedance and change with time of the skin 6. Such control can be performed in accordance with, for example, the following multivariate control.

Let I₉₁, I₉₂, I₉₃, I₉₄, and I₉₅ denote actual values for the currents in the respective load resistances 91, 92, 93, 94, and 95, and let V₉₁, V₉₂, V₉₃, V₉₄, and V₉₅ denote actual values for the voltages in the resistances. When a current vector Iᵢ=(I₉₁, I₉₂, I₉₃, I₉₄, I₉₅) and a voltage vector Vᵢ=(V₉₁, V₉₂, V₉₃, V₉₄, V₉₅) are defined, an expression (1) (Iᵢ=MA+MB×Vᵢ) is established. In the expression, MA denotes a matrix showing the internal state of a system independent of Vᵢ, and MB denotes a matrix showing each of a skin resistance and the internal resistance of an iontophoresis device against an ionic drug. In addition, MA and MB are estimated from Iᵢ and Vᵢ to be sequentially measured with the current detection circuits and from the expression (1). A control voltage Vᵢ for realizing a preset current value Iᵢ is calculated from the estimated MA and MB, and from an expression (2) (Vᵢ=Inv(MB) (Iᵢ−MA)) derived from the expression (1). The CPU 12 outputs a feedback signal for realizing the control voltage Vᵢ thus determined, and performs control in such a manner that a current having a defined value finally flows into each electrode assembly. Therefore, according to one embodiment of the present invention, the current control means in the iontophoresis device performs control in such a manner that a current having a defined value flows into an electrode assembly.

In addition, the following conditions may, for example, be adopted as energizing conditions in the iontophoresis device 1.

(1) Constant current condition, for example, 0.1 to 0.5 mA/cm², or 0.1 to 0.3 mA/cm²

(2) Safe voltage condition that realizes the above constant current, for example, 50 V or less, or 30 V or less

According to embodiments of the present invention, the total number of electrode assemblies 21, 22, 23, 24, and 25, and a combination of the number of first electrode assemblies 21, 22, and 23 and the number of second electrode assemblies 24 and 25 are not limited to the above example. Embodiments of the present invention can be embodied even when these numbers are appropriately changed. It is easy for one skilled in the art to conceive a constitution with those numbers appropriately changed from the above example. For example, the number of the electrode assemblies 21, 22, 23, 24, and 25 can be increased or reduced by increasing or reducing the transistors 81, 82, 83, 84, and 85, the load resistances 91, 92, 93, 94, and 95, the current detection circuits 101, 102, 103, 104, and 105, and the like.

According to one embodiment, the ionic drugs to be held by the respective electrode assemblies 21, 22, 23, 24, and 25 in the iontophoresis device 1 are a sleep-inducing agent and a stimulant. It should be noted that each of the sleep-inducing agent and the stimulant may be a combination of multiple drugs.

For the ionic drugs to be held by at least one of the electrode assemblies 21, 22, 23, 24, and 25, examples of the sleep-inducing agent capable of being positively ionized may include the following.

Narcotic: barbital, amobarbital, bromvalerylurea, rilmazafone hydrochloride, flunitrazepam, flurazepam hydrochloride, triazolam, midazolam, brotizolam, estazolam, lormetazepam, zopiclone, quazepam.

Tranquilizer: lithium carbonate, carbamazepine.

Antidepressant: nortriptyline hydrochloride, amoxapine, maprotiline hydrochloride, imipramine hydrochloride, amitriptyline hydrochloride, trimipramine maleate, clomipramine hydrochloride, lofepramine hydrochloride, dosulepin hydrochloride, trazodone hydrochloride, fluvoxamine maleate, paroxetine hydrochloride hydrate, milnacipran hydrochloride, mianserin hydrochloride, setiptiline maleate, tandospirone citrate.

Antianxiety: etizolam, clotiazepam, alprazolam, flutazolam, lorazepam, fludiazepam, bromazepam, mexazolam, diazepam, cloxazolam, chlordiazepoxide, prazepam, hydroxyzine.

Antihistaminic agent: diphenhydromine hydrochloride, diphenylpyraline hydrochloride, diphenylpyraline teoclate, clemastine fumarate, chlorpheniramine maleate, triprolidine hydrochloride, alimemazine tartrate, promethazine hydrochloride, homochlorcyclizine hydrochloride, cyproheptadine hydrochloride.

Examples of the sleep inducing agent capable of being negatively ionized may include the following.

Narcotic: pentobarbital calcium, phenobarbital sodium, secibarbital sodium, chloral hydrate.

Antidepressant: clorazeptate dipotassium, ethyl loflazepate.

On the other hand, examples of the stimulant capable of being positively ionized include the following.

Analeptic drug: caffeine, methamphetamine hydrochloride, methylphenidate hydrochloride, pemoline, fursultiamine.

Antidepressant: tandospirone citrate.

Cerebral circulation activator: citicoline, adenosine triphosphate disodium, meclofenoxate hydrochloride, tiapride hydrochloride, ifenprodil tartrate, nicergoline, ibudilast, dihydroergotixine mesilate, nizofenone fumarate, fasudil hydrochloride, norepinephrine.

An example of the stimulant capable of being negatively ionized may include the following.

Cerebral circulation activator: gamma-aminobutyric acid, calcium hopantenate.

Examples of main side effects of a sleep-inducing agent may include: poor awakening and dull feeling during the daytime due to the transfer of an effect of a narcotic up to the time of rising or any time after the rising; drift and weakness due to the muscle-relaxing effect possessed by the narcotic; and headache dull and malaise due to the transfer of an effect of the narcotic up to any time after rising. Administering a stimulant upon or immediately before awakening can considerably alleviate those side effects.

In addition, an inactive electrode made of a conductive material such as carbon or platinum can be used as an electrode of the electrode assembly 21, 22, 23, 24 or 25. The electrolyte solution holding portion 212 may be constituted by a thin film that has the property of holding an electrolyte solution by being impregnated with the electrolyte solution. The thin film can be made of the same material as that used for a drug solution holding portion 214 used for holding an ionic drug by being impregnated with the ionic drug, as described later.

A desired one can be appropriately used as the electrolyte solution depending upon the conditions such as a drug to be applied. However, an electrolyte solution that damages the skin 6 of the organism 14 owing to an electrode reaction should be avoided. An organic acid or a salt thereof present in a metabolic cycle of the organism 14 may be advantageous as the electrolyte solution in one embodiment of the present invention in terms of harmlessness. For example, lactic acid and fumaric acid may be used. In particular, an aqueous solution of 1M of lactic acid and 1M of sodium fumarate (1:1) may be used. Such electrolyte solution may be used because: it has high solubility with respect to water and passes a current well; and in the case where a current is allowed to flow at a constant level, the electric resistance is low and a change in pH is relatively small in an electric power source device.

A cation exchange membrane and an anion exchange membrane may be used together as ion exchange membranes 213, 215 to be used for the electrode assembly 21, 22, 23, 24, and 25. Some examples of the cation exchange membrane include NEOSEPTAs (CM-1, CM-2, CMX, CMS, CMB, and CLE04-2) manufactured by Tokuyama Corporation. Some examples of the anion exchange membrane include NEOSEPTAs (AM-1, AM-3, AMX, AHA, ACH, ACS, ALE04-2, and AIP-21) manufactured by Tokuyama Corporation. Other examples may include: a cation exchange membrane that includes a porous film having cavities a part or whole of which are filled with an ion exchange resin having a cation exchange function; and an anion exchange resin that includes a porous film having cavities a part or whole of which are filled with an ion exchange resin having an anion exchange function.

The above-mentioned ion exchange resins can be fluorine-based that include a perfluorocarbon skeleton having an ion exchange group and hydrocarbon-based ones that include a nonfluorinated resin as a skeleton. From the viewpoint of convenience of production process, hydrocarbon-based ion exchange resins may be used. The filling rate of the porous film with the ion exchange resin, which varies depending on the porosity of the porous film, may, for example, be 5 to 95 mass %, 10 to 90 mass %, or 20 to 60 mass %.

The ion exchange group in the above-mentioned ion exchange resin is not particularly limited in so far as it may be a functional group that generates a group having negative or positive charge in aqueous solutions. Such functional group may be present in the form of a free acid or a salt. Examples of a cation exchange group include a sulfonic group, a carboxylic acid group, and a phosphonic acid group. Of those, a sulfonic group may be used. Examples of a counter cation for the cation exchange group include: alkali cations such as a sodium ion and a potassium ion; and ammonium ions. Examples of an anion exchange group include a primary amino group, a secondary amino group, a tertiary amino group, a quaternary ammonium group, a pyridyl group, an imidazole group, a quaternary pyridium group, and a quaternary imidazolium group. Of those, a quaternary ammonium group or a quaternary pyridium group may be used. Examples of a counter cation for the anion exchange group include: halogen ions such as a chlorine ion; and hydroxy ions.

The above-mentioned porous film is not particularly limited and any porous film may be used in so far as it is in the form of a film or sheet that has a large number of pores communicating with both sides thereof. To satisfy both of high strength and flexibility, it may be advantageous that the porous film be made of a thermoplastic resin. Examples of the thermoplastic resin constituting the porous film include: polyolefin resins such as homopolymers or copolymers of α-olefins such as ethylene, propylene, 1-butene, 1-pentene, 1-hexene, 3-methyl-1-butene, 4-methyl-1pentene, and 5-methyl-1-heptene; vinyl chloride-based resins such as polyvinyl chloride, vinyl chloride-vinyl acetate copolymers, vinyl chloride-vinylidene chloride copolymers, and vinyl chloride-olefin copolymers; fluorine-based resins such as polytetrafluoroethylene, polychlorotrifluoroethylene, polyvinylidene fluoride, tetrafluoroethylene-hexafluoropropylene copolymers, tetrafluoroethylene-perfluoroalkyl vinylether copolymers, and tetrafluoroethylene-ethylene copolymers; polyamide resins such as nylon 66; and polyimide resins. Of those, polyolefin resins may be advantageously used in consideration of, for example, mechanical strength, flexibility, chemical stability, and chemical resistance. Of those, polyethylene or polypropylene may be used.

The properties of the above-mentioned porous film made of the thermoplastic resin are not particularly limited. The mean pore size may be 0.005 μm to 5.0 μm, 0.01 μm to 2.0 μm, or 0.02 μm to 0.2 μm in consideration of the formation of the ion exchange membrane 213, 215, 243 that is thin and has excellent strength and low electric resistance. The above-mentioned mean pore size as used herein may be a mean flow pore size measured in conformance with a bubble point method (e.g., JIS K3832-1990). Similarly, the porosity of the porous film may, for example, be 20% to 95%, 30% to 90%, or 30% to 60%. In consideration of the thickness of the ion exchange membrane 213, 215, 243 to be formed, the thickness of the porous film may be 5 μm to 140 μm, 10 μm to 130 μm, or 15 μm to 55 μm. An anion exchange membrane or a cation exchange membrane formed of such porous film may have same thickness as that of the porous film or up to about 20 μm larger than the thickness of the porous film.

Furthermore, the drug solution holding portion 214 may comprise a thin film that holds an ionic drug by being impregnated with the ionic drug or the like. It may be advantageous for the thin film to hold the ionic drug by being impregnated with the ionic drug or the like, and to cause an ionized drug impregnated therein to move (i.e., ion transferability or ion conductivity) toward the skin 6 under defined electric field conditions. Examples of materials that sufficiently combine properties of holding a drug by being impregnated therein and ion conductivity include hydrogel forms of acrylic resins (e.g., acrylic hydrogel film), a segmented polyurethane-based gel film, and an ion-conductive porous sheet for forming a gel-like solid electrolyte (e.g., a porous polymer disclosed in JP 11-273452 A using, as a base, an acrylonitrile copolymer containing 50 mol % or more, 70 mol % to 98 mol % or more of acrylonitrile and having a porosity of 20% to 80%). When such drug solution holding portion 214 as described above is impregnated with a drug, an impregnation rate (defined, for example, by 100×(W-D)/D [%] where D indicates a dry weight and W indicates a weight after impregnation) may be 30% to 40%.

When multiple electrode assemblies 21, 22, 23, 24 or 25 are gathered in one package so that the drug administration means 2 may be constituted integrally, any material can be used for the package without any particular limitation as long as the material does not affect the administration of the ionic drug. For example, polyolefin for medical equipment, and the like may be used.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to WO 03/037425 A1 are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An iontophoresis device comprising:
an electric power source device;
drug administration device including at least two or more electrode assemblies each holding an ionic drug, the drug administration device coupled to the electric power source device; and
current control device to control current flowing to respective ones of the electrode assemblies,
an amount of the ionic drug is releasable from each of the electrode assemblies at a defined time when transdermally administered to an organism in accordance with the current flowing from the current control device, wherein
at least one of the two or more electrode assemblies holds a sleep-inducing agent as the ionic drug, and at least another one of the two or more electrode assemblies holds a stimulant as the ionic drug.

2. The iontophoresis device according to claim 1, wherein the electrode assemblies of the drug administration device comprises:
two or more first electrode assemblies to respectively hold the ionic drug; and
one or more second electrode assemblies to serve as counter electrodes of the first electrode assemblies.

3. The iontophoresis device according to claim 1, wherein the drug administration device includes at least four or more electrode assemblies having:
a first plurality of first electrode assemblies respectively holding the ionic drug;
a first plurality of second electrode assemblies respectively holding the ionic drug;
a second plurality of second electrode assemblies to serve as counter electrodes of the first plurality of first electrode assemblies; and
a second plurality of first electrode assemblies to serve as counter electrodes of the first plurality of second electrode assemblies.

4. The iontophoresis device according to claim 2, wherein each of the two or more first electrode assemblies comprises:
a first electrode coupled to the electric power source device having same polarity as that of a drug component of the ionic drug in the first electrode assembly;
a first electrolyte solution holding portion to hold an electrolyte solution by being impregnated with the electrolyte solution, the first electrolyte solution holding portion positioned adjacent the first electrode;
a first ion exchange membrane to substantially pass ions having a polarity that is same as a polarity of the ionic drug and substantially block ions having a polarity that is opposite the polarity of the ionic drug, the first ion exchange membrane positioned adjacent the electrolyte solution holding portion;
a drug solution holding portion to hold the ionic drug by being impregnated with the ionic drug, the drug solution holding portion positioned adjacent the first ion exchange membrane; and
a second ion exchange membrane to substantially pass ions having a polarity opposite the polarity of the ionic drug and substantially block ions having a polarity that is same as the polarity of the ionic drug, the second ion exchange membrane positioned adjacent the drug solution holding portion.

5. (canceled)

6. The iontophoresis device according to claim 1, wherein the drug administration device is configured integrally.

7. The iontophoresis device according to claim 1, wherein the current control device comprises:
a load resistor provided between each of the electrode assemblies and the electric power source device;
a current detecting portion to detect a current flowing to the load resistor; and
a feedback control portion to allow a controlled current to flow to each of the electrode assemblies in accordance with an output from the current detecting portion.

8. A method of operating an iontophoresis device comprising:
providing an electric power source device;
coupling a drug administration device to the electric power source device, the drug administration device including at least two or more electrode assemblies respectively holding a sleep-inducing drug and a stimulant drug;
placing the drug administration device on a skin surface of an organism;
energizing the drug administration device with the electric power source device;
providing a current control device to control current flowing through respective ones of the electrode assemblies; and
releasing a defined amount of the sleep-inducing drug and the stimulant drug at a defined time.

9. The iontophoresis device of claim 2, wherein each of the one or more second electrode assemblies comprises:
a second electrode electrically coupled to the electric power source device to have a polarity opposite that of the first electrode in each of the two or more first electrode assemblies;
a second electrolyte solution holding portion to hold an electrolyte solution by being impregnated with the electrolyte solution, the electrolyte solution holding portion positioned adjacent the second electrode; and
a third ion exchange membrane to substantially pass ions having a polarity that is same as a polarity of the ionic drug and substantially block ions having a polarity that is opposite the polarity of the ionic drug, the third ion exchange membrane positioned adjacent the second electrolyte solution holding portion.
